(19) United States
(12) Patent Application Publication
(10) Pub. No.: US 2009/0043367 A1
(43) Pub. Date: Feb. 12, 2009

(54) APPARATUS AND METHODS FOR REMOVING AN ELECTRONIC IMPLANT FROM A BODY

(76) Inventors: Yitzhak ZILBERMAN, Santa Clarita, CA (US); Mark Chamberlain, Santa Claria, CA (US); Arkady Glukhovsky, Santa Clarita, CA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 12/187,655

(22) Filed: Aug. 7, 2008

Related U.S. Application Data
(60) Provisional application No. 60/954,838, filed on Aug. 9, 2007.

(51) Int. Cl. A61N 1/375

(52) U.S. Cl. 607/116

(57) ABSTRACT

An apparatus includes an electronic stimulator configured to be implanted within a body, and a flexible member coupled to the electronic stimulator by an adhesive. In some embodiments, the flexible member is formulated to be soluble when exposed to a bodily tissue.

### CROSS-REFERENCE TO RELATED APPLICATIONS

This application claims priority to U.S. Provisional Application Ser. No. 60/954,838, entitled “Assembly for Facilitating Removal of Miniature Implantable Device, and Related Method for Making It,” filed Aug. 9, 2007, which is incorporated herein by reference in its entirety.

### BACKGROUND

The invention relates generally to medical devices and procedures, and more particularly to apparatus and methods for removing an electronic implant from a body via a member disposed outside of the body.

Electronic implants, such as, for example, microstimulators, electrical stimulation leads and/or electrical sensing leads, are used in various medical procedures. For example, some known electronic implants can be implanted within a body to stimulate a response from a bodily organ or tissue, such as, for example, the heart, a nerve, a muscle group or the like. Other known electronic implants can be implanted within a patient's body to sense a response from a bodily organ or tissue. Accordingly, known electronic implants are often implanted into the patient's body at a predetermined location and/or orientation (e.g., such that a portion of the electronic implant is in contact with a nerve).

In certain instances, an electronic implant may need to be moved within and/or removed from the body after it has implanted. For example, in some instances, an electronic implant may not be disposed at the desired location and/or orientation within the body. Some known procedures for removing electronic implants include reopening the incision through which the electronic implant was inserted and/or forming a new incision adjacent the implant. Other known procedures for removing electronic implants include attaching a tether to the electronic implant via a coupling member, such as, for example, an eyelet. Such coupling members can be difficult to attach to the electronic implant, and can increase the size of and/or decrease the electronic performance of the electronic implant.

Thus, a need exists for improved apparatus and methods for moving an electronic implant within and/or removing an electronic implant from a body.

### SUMMARY

Apparatus and methods for moving an electronic implant within a body are described herein. In some embodiments, an apparatus includes an electronic stimulator configured to be implanted within a body, and a flexible member coupled to the electronic stimulator by an adhesive. In some embodiments, the flexible member is formulated to be soluble when exposed to a bodily tissue.

### BRIEF DESCRIPTION OF THE DRAWINGS

FIGS. 1-3 are schematic illustrations of an electronic stimulator according to an embodiment disposed within a body in a first configuration, a second configuration, and a third configuration, respectively.

FIG. 4 is a flow chart of a method of moving an electronic implant within the body according to an embodiment.

FIGS. 5-7 are schematic illustrations showing the method of moving the electronic implant within the body according to the embodiment shown in FIG. 4.

FIG. 8 is a schematic illustration of an electronic stimulator according to an embodiment.

FIG. 9 is a schematic illustration of an enclosure for an electronic stimulator according to an embodiment.

FIG. 10 is a flow chart of a method of disposing a coupling member about an electronic stimulator according to an embodiment.

FIGS. 11-12 are schematic illustrations showing the method of disposing a coupling member about an electronic stimulator according to the embodiment shown in FIG. 10.

### DETAILED DESCRIPTION

In some embodiments, an apparatus includes an electronic stimulator and a flexible member. The electronic stimulator, which can be, for example, a BION® microstimulator manufactured by Boston Scientific Neuromodulation, is configured to be implanted within a body. The flexible member is coupled to the electronic stimulator by an adhesive. In some embodiments, the flexible member can be coupled to an outer surface of the electronic stimulator. In some embodiments, the flexible member is formulated to be soluble when exposed to a bodily tissue. In some embodiments, the adhesive can be formulated to be soluble when exposed to the bodily tissue.

In some embodiments, a method includes inserting at least a distal end portion of an insertion tool within a body such that a stimulation portion of an electronic implant is disposed within the body and at least a proximal portion of the electronic implant is disposed outside the body. The proximal portion of the electronic implant is soluble in a bodily tissue, and is coupled to the stimulation portion by an adhesive. The insertion tool is removed from the body after being inserted such that the stimulation portion of the electronic implant is at a first location within the body. The proximal portion of the electronic implant is moved from a region outside the body such that the stimulation portion is moved to a second location within the body. The second location of the stimulation portion is different from the first location.

In some embodiments, an apparatus includes an enclosure and a flexible member. The enclosure is configured to be disposed about an electronic stimulator when the electronic stimulator is disposed within a body. The enclosure is constructed from at least one filament formulated to be soluble when exposed to a bodily tissue, such as, for example, a catgut suture, a suture constructed from polyglycolic acid, or the like. The flexible member is coupled to the enclosure, and has a length such that a proximal end portion of the flexible member is disposed outside of the body when the electronic stimulator is disposed within the body.

In some embodiments, a method includes disposing a coupling member about a portion of an electronic stimulator. The coupling member can be, for example, a cap having an eyelet to which a flexible member can be attached. The coupling member defines an opening having a size greater than a size of the portion of the electronic stimulator. The size of the opening of the coupling member is changed such that the size of the opening of the coupling member is less than the size of the portion of the electronic stimulator.

As used in this specification, the words “proximal” and “distal” can refer to the direction closer to and away from, respectively, an operator (e.g., surgeon, physician, nurse, technician, etc.) who would use a medical device or a therapeutic device during a procedure. For example, the end of a medical device first to contact the patient's body would be the distal end, while the opposite end of the medical device (e.g., the end of the medical device being operated by the operator) would be the proximal end of the medical device. Similarly, the end of a medical device implanted the furthest within the patient's body would be the distal end, while the opposite end of the medical device (e.g., the end of the medical device that is implanted the least amount within the body or the end of the medical device that is disposed outside of the body) would be the proximal end.

As used in this specification the words “electronic stimulator” and “electronic implant” can refer to any object or device that can be used as a part of an electrical circuit or an electrical process associated with electronically stimulating a bodily tissue. For example, in some embodiments, an electronic stimulator and/or an electronic implant can include passive objects, such as conductive wires, passive switches, insulators, electrical connectors or the like. In other embodiments, an electronic stimulator and/or an electronic implant can include an electrical device that produces, processes, receives and/or otherwise manipulates an electrical signal. Such electrical devices can include, for example, signal processors, sensors, stimulators, or the like.

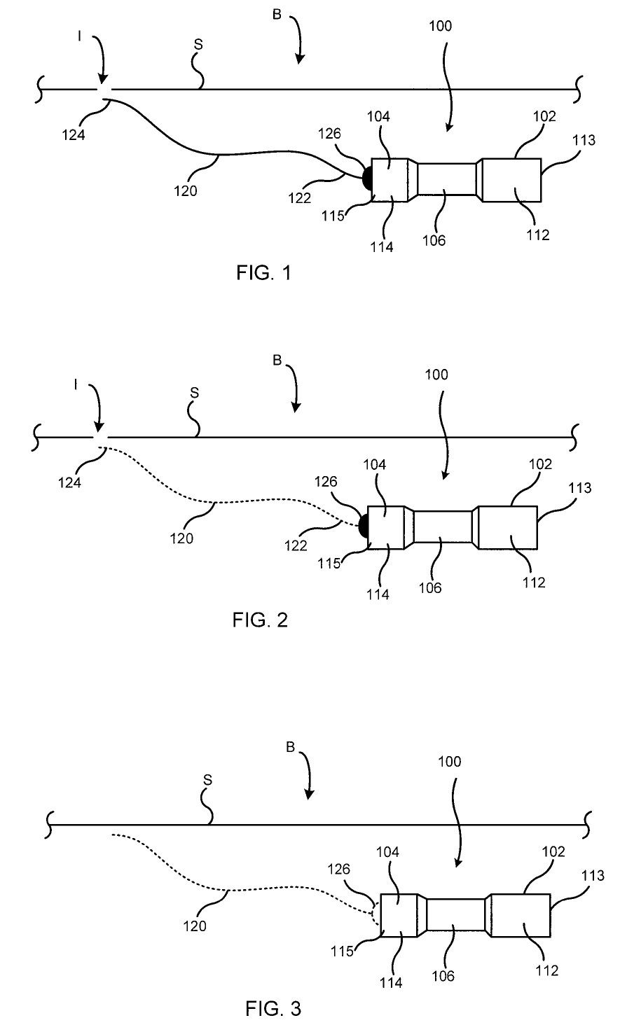

FIGS. 1-3 are schematic illustrations of an electronic stimulator 100 according to an embodiment of the invention disposed within a body B in a first configuration, a second configuration, and a third configuration, respectively. The electronic stimulator 100 can be, for example, an implantable stimulator of the types shown and described in U.S. Pat. No. 5,324,316, entitled “Implantable Microstimulator,” filed Mar. 3, 1993, and U.S. Pat. No. 6,735,474, entitled “Implantable Stimulator System and Method for Treatment of Incontinence and Pain,” filed Aug. 18, 2000, each of which is incorporated herein by reference in its entirety. In some embodiments, for example, the electronic stimulator 100 can be a BION® implantable microstimulator manufactured by Boston Scientific Neuromodulation, a radio frequency-powered implantable microstimulator developed by the Alfred Mann Foundation, a radio frequency-powered implantable microstimulator developed by the Alfred Mann Institute, or the like.

The electronic stimulator 100 has a distal end portion 102, a proximal end portion 104 and a central portion 106. The distal end portion 102 includes a distal end electrode 112 having an outer surface 113. The distal end electrode 112 can be either a positively-charged electrode (i.e., an anode) or a negatively-charged electrode (i.e., a cathode). The proximal end portion 104 includes a proximal end electrode 114 having an outer surface 115. The proximal end electrode 114 can be either a positively-charged electrode (i.e., an anode) or a negatively-charged electrode (i.e., a cathode). The central portion 106 is disposed between the distal end electrode 112 and the proximal end electrode 114 and in some embodiments, can electronically isolate the distal end electrode 112 and the proximal end electrode 114. The central portion 106 can include, for example, a glass or ceramic portion. In this manner, when the electronic stimulator 100 is disposed within the body B, an electronic current (not shown in FIGS. 1-3) can travel between the distal end electrode 112 and the proximal end electrode 114 to stimulate a target location such as a muscle, a nerve or the like.

A flexible member 120 is coupled to the electronic stimulator 100 by an adhesive 126. More particularly, a distal end portion 122 of the flexible member 120 is coupled to the outer surface 115 of the proximal end electrode 114 of the electronic stimulator 100, which can be either the cathode electrode or the anode electrode, by the adhesive 126. As shown in FIG. 1, the electronic stimulator 100 can be implanted into the body B via an incision I in the skin S when the flexible member 120 is coupled to the electronic stimulator 100. Moreover, the flexible member 120 has a length such that a proximal end portion 124 of the flexible member 120 can be disposed beneath the skin S adjacent the incision I when the electronic stimulator 100 is disposed within the body B. As described in more detail herein, this arrangement allows a user to move the electronic stimulator 100 within the body B via the flexible member 120. In some embodiments, a user can remove the electronic stimulator 100 from the body B via the flexible member 120.

The flexible member 120 is constructed from a material formulated to be soluble when the flexible member 120 is exposed to a bodily tissue (e.g., a bodily fluid). Such bodily tissues can include, for example, blood, mucous, water, saliva, urine, fat, muscle tissue, or the like. Similarly stated, the flexible member 120 is configured to dissolve after a being disposed within the body B. Said another way, the flexible member 120 is configured to be broken down and/or metabolized by the body B after a being disposed within the body B. The flexible member 120 can be constructed from any suitable biocompatible material formulated to be soluble when exposed to a bodily tissue. In some embodiments, for example, the flexible member 120 can be constructed from a natural material, such as cutgut (e.g., sheep or bovine intestines), chromic catgut (i.e., twisted collagen strands), or the like. In other embodiments, the flexible member 120 can be constructed from a synthetic material, such as polyglycolic acid, polydioxanone, polylactic acid, caprolactone, or the like.

The adhesive 126 can be any suitable biocompatible adhesive. Although the adhesive 126 is shown and described below as being soluble when the adhesive 126 is exposed to a bodily tissue of the types described above, in other embodiments, the adhesive 126 can be non-soluble when exposed to a bodily tissue. In some embodiments, the adhesive 126 can be a soluble adhesive, such as, for example, a fibrin glue (which includes fibrinogen and thrombin), BioGlue surgical adhesive, produced by CryoLife Inc., or the like. In other embodiments, the adhesive can be a non-soluble adhesive, such as, for example, light-curing acrylics and light-curing cyanoacrylates, light-curing silicones, cyanoacrylate adhesives, epoxy adhesives, and polyurethane adhesives.

FIG. 2 shows the electronic stimulator 100 within the body B in the second configuration, after the electronic stimulator 100 has been within the body B a first predetermined time period. When the electronic stimulator 100 is in the second configuration, the flexible member 120 is dissolved within the body B. The flexible member 120 is shown as a dashed line in FIG. 2 to indicate that the flexible member 120 has been dissolved within the body B. In this manner, the flexible member 120 can be dissolved after the first predetermined time period such that the flexible member 120 will not cause irritation, infection or the like. The first predetermined time period can be any suitable time period. For example, in some embodiments, the first predetermined time period can be a period of time during which a user may desire to move the electronic stimulator 100 via the flexible member 120. In some embodiments, the first predetermined time period can be associated with the time period during which the electronic stimulator 100 can become encapsulated (e.g., surrounded) by bodily tissue such that movement of the electronic stimulator 100 via the flexible member 120 is not desirable. In some embodiments, for example, the first predetermined time period can be less than approximately 14 days. In other embodiments, for example, the first predetermined time period can be less than approximately 21 days. In yet other embodiments, the first predetermined time period can be between approximately 8 days and 14 days. In yet other embodiments, the first predetermined time period can be between approximately 14 days and 21 days. In yet other embodiments, the first predetermined time period can be approximately 14 days.

FIG. 3 shows the electronic stimulator 100 within the body B in the third configuration, after the electronic stimulator 100 has been within the body B a second predetermined time period. In some embodiments, the second predetermined time period can end substantially simultaneously with the first predetermined time period (i.e., the first predetermined time period and the second predetermined time period are substantially equal). In other embodiments, the second predetermined time period can end after the first predetermined time period (i.e., the second predetermined time period is longer than the first predetermined time period). When the electronic stimulator 100 is in the third configuration, the flexible member 120 and the adhesive 126 are dissolved within the body B. The flexible member 120 and the adhesive 126 are shown as a dashed line in FIG. 3 to indicate that the flexible member 120 and the adhesive 126 have been dissolved within the body B. In this manner, the adhesive 126 can be dissolved after the second predetermined time period such that the flexible member 120 and the adhesive 126 will not cause irritation, infection or the like. The second predetermined time period can be any suitable time period. For example, in some embodiments, the second predetermined time period can be a period of time during which a user may desire to move the electronic stimulator 100 via the flexible member 120. In some embodiments, the second predetermined time period can be associated with the time period during which the electronic stimulator 100 can become encapsulated (e.g., surrounded) by bodily tissue such that movement of the electronic stimulator 100 via the flexible member 120 is not desirable. In some embodiments, for example, the second predetermined time period can be less than approximately 14 days. In other embodiments, for example, the second predetermined time period can be less than approximattely 21 days. In yet other embodiments, the second predetermined time period can be between approximately 8 days and 14 days. In yet other embodiments, the second predetermined time period can be between approximately 14 days and 21 days. In yet other embodiments, the second predetermined time period can be approximately 14 days.

In some embodiments, the flexible member 120 and the adhesive 126 can be collectively configured to withstand a tensile force sufficient to move the electronic stimulator 100 within the body B and/or remove the electronic stimulator 100 from the body B. Similarly stated, in some embodiments, the flexible member 120 and the adhesive 126 can be collectively configured to remain intact and coupled to the electronic stimulator 100 when a force is applied to the proximal end portion 124 of the flexible member 120 sufficient to move the electronic stimulator 100 within the body B. In this manner, the user can pull the proximal end portion 124 of the flexible member 120 with sufficient force to move the electronic stimulator 100 within the body B and/or remove the electronic stimulator 100 from the body B. In some embodiments, for example, the flexible member 120 and the adhesive 126 can be collectively configured to withstand a tensile force of at least 1.8 N (0.4 lbf). In other embodiments, the flexible member 120 and the adhesive 126 can be collectively configured to withstand a tensile force of at least 3.6 N (0.8 lbf).

Although the flexible member 120 is shown and described above as being coupled to the proximal end portion 104 of the electronic stimulator 100, in other embodiments, a flexible member can be coupled to any suitable location of the electronic stimulator 100. For example, in some embodiments, the flexible member 120 can be coupled to the end surface of the proximal end portion 104. In this manner, the addition of the flexible member 120 does not increase the profile (i.e., the maximum size or the outer diameter) of the electronic stimulator 100. Thus, the electronic stimulator 100 can be inserted into the body B using the same insertion tools as used for inserting electronic stimulators that do not include a flexible member. In other embodiments, the flexible member 120 can be coupled to the circumferential surface of the electronic stimulator 100 (e.g., the flexible member 120 can be wrapped about the circumference of the electronic stimulator 100).

Although the electronic stimulator 100 is shown as including a distal end electrode 112 and a proximal end electrode 114, in some embodiments, an electronic stimulator can be any suitable electrical device configured to convey an electronic signal (e.g., a current) within the body to a target location. For example, in some embodiments, an electronic stimulator can include a terminal (or pick-up) portion, a stimulation portion (e.g., a cuff electrode, an exposed electrical conductor or the like), and a flexible conductor disposed therebetween. Such an electronic stimulator can be used in the stimulation systems shown and described in U.S. Patent Publication No. 2006/0184211, entitled “Method of Routing Electrical Current to Bodily Tissues Via Implanted Passive Conductors,” filed Jan. 23, 2006, which is incorporated herein by reference in its entirety.

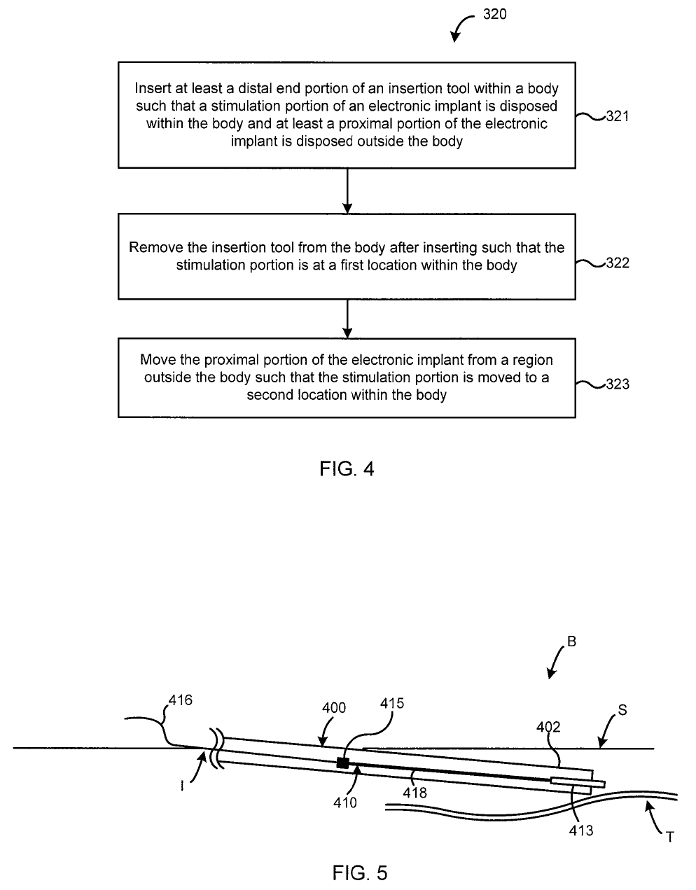

For example, FIG. 4 is a flow chart of a method 320 of moving an electronic implant within a body via a proximal portion of the electronic implant according to an embodiment of the invention. The method illustrated in FIG. 4 is discussed with reference to FIGS. 5-7, which are schematic illustrations of an electronic implant 410 disposed within a body B in a first configuration, a second configuration and a third configuration, respectively. The method includes inserting at least a distal end portion of an insertion tool within a body such that a stimulation portion of an electronic implant is disposed within the body and at least a proximal portion of the electronic implant is disposed outside the body, 321. Referring to FIG. 5, at least a distal end portion 402 of an insertion tool 400 is inserted into the body B of a patient through a skin incision I. The electronic implant 410 is coupled to the insertion tool 400 such that a stimulation portion 413 of the electronic implant 410 is inserted into the body B along with the distal end portion 402 of the insertion tool 400. In some embodiments, the insertion tool 400 defines a lumen (not shown) within which the electronic implant 410 can be substantially housed, as described above.

The electronic implant 410 includes the stimulation portion 413, a terminal portion 415, a conductor 418, and a proximal portion 416. The conductor 418, which can be constructed of a substantially flexible material, is disposed between the stimulation portion 413 and the terminal portion 415. The proximal portion 416 of the electronic implant 410 is coupled to the stimulation portion 413. The proximal portion 416 of the electronic implant 410 is constructed of a material that is soluble when exposed to a bodily tissue. Such materials can be flexible or rigid, and can include, for example, polyglycolic acid (PGA), polylactic acid, collagen, polycaprolactone, hylauric acid, polyethylene glycol, polyvinylpyrrolidone, high molecular weight carbohydrates, hydroxypropylcellulose and/or any combination thereof. In some embodiments, the proximal portion 416 is configured to substantially dissolve within a predetermined time (e.g., approximately 7 days, approximately 14 days, approximately 21 days, or the like) of being in contact with a bodily tissue. The proximal portion 416 of the electronic implant 410 can be any length and can be used to move the electronic implant 410 within the body B, as described herein.

As shown in FIG. 5, the distal end portion 402 of the insertion tool 400 is inserted into the body B such that at least a portion of the proximal portion 416 of the electronic implant 410 is disposed outside the body B, the terminal portion 415 is disposed beneath the skin S, and the stimulation portion 413 is disposed adjacent a target location T within the body B. The target location T can be, for example, a median nerve. In some embodiments, the stimulation portion 413 can be disposed apart from the target location T. In other embodiments, the stimulation portion 413 can be in contact with the target location T.

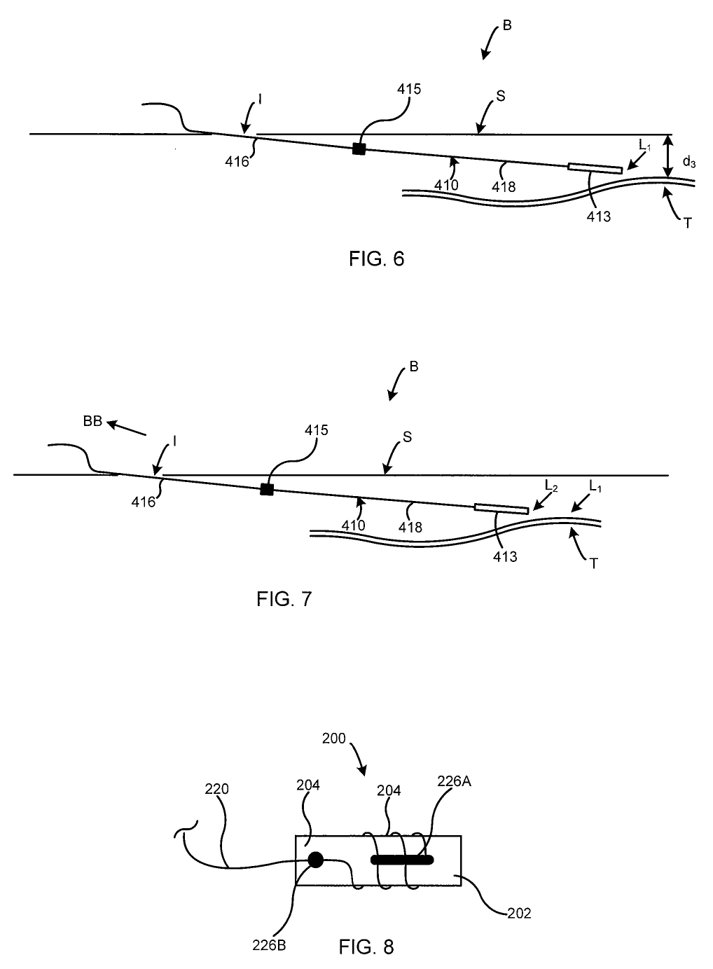

Returning to the flow chart shown in FIG. 4, the insertion tool is removed from the body such that the stimulation portion is at a first location within the body, 322. As shown in FIG. 6, the insertion tool 400 is removed from the body B through the skin incision I such that the stimulation portion 413 of the electronic implant 410 remains in the body B disposed beneath the skin S at a predetermined depth, d₃, and at a location L₁. The location L₁ of the stimulation portion 413 of the electronic implant 410 is adjacent the target location T. In some embodiments, however, the stimulation portion 413 of the electronic implant 410 can be in contact with the target location T such that the location L₁ of the stimulation portion 413 is the point of contact between the target location T and the stimulation portion 413. As shown in FIG. 6, at least a proximal portion 416 of the electronic implant 410 remains disposed outside the body B when the insertion tool 400 is removed.

Returning to the flow chart shown in FIG. 4, the proximal portion of the electronic implant is moved from a region outside the body such that the stimulation portion is moved to a second location within the body, 323. As shown in FIG. 7, the proximal portion 416 of the electronic implant is moved in a direction BB from a region outside the body B. More particularly, a user, such as, for example, a surgeon, can exert a force on the proximal portion 416 of the electronic implant 410 such that the electronic implant 410 moves within the body B. As a result, the stimulation portion 413 is moved within the body B, in the direction BB, from the first location L₁ to a second location L₂. In some embodiments, the direction BB can be substantially opposite the direction in which the electronic implant 410 was inserted. In this manner, the surgeon can move the stimulation portion 413 of the electronic implant 410 within the body B without performing additional operations and/or making additional incisions.

In other embodiments, the flexible member 120 can be coupled to the central portion 106 of the electronic implant. For example, FIG. 8 is a schematic illustration of an electronic stimulator 200 according to an embodiment of the invention. The electronic stimulator 200 has a distal end portion 202, a proximal end portion 204 and a central portion 206. In some embodiments, the distal end portion 202 and the proximal end portion 204 can each include an electrode, as described above. The central portion 206 is disposed between the distal end portion 202 and the proximal end portion 204, and in some embodiments, can electronically isolate the distal end portion 202 and the proximal end portion 204. The central portion 206 can include, for example, a glass or ceramic portion.

A flexible member 220 is disposed about and coupled to the central portion 206 of the electronic stimulator 200. More particularly, the flexible member 220 is wrapped about the outer surface (e.g., the circumference) of the central portion 206 of the electronic implant 200. In this manner, the coupling between the flexible member 220 and the electronic stimulator 200 can be enhanced by the frictional force resulting from the wrapping of the flexible member 220 about the electronic stimulator 200. Although FIG. 8 shows the flexible member 220 being wrapped about the outer surface of the central portion 206 by approximately three full revolutions (i.e., 1080 degrees), in other embodiments, the flexible member 220 can be wrapped about any portion of the electronic implant 200 any suitable number of revolutions. For example, in some embodiments, the flexible member 220 can be wrapped about the outer surface of the central portion 206 of the implant 200 by at least two full revolutions (i.e., 720 degrees). In other embodiments, the flexible member 220 can be wrapped about the outer surface of the central portion 206 of the implant 200 by between two and five revolutions.

The flexible member 220 is coupled to the electronic stimulator 200 by an adhesive 226. In some embodiments, the adhesive 226 can be placed about the surface of the electronic stimulator 200 in discrete locations, which are labeled as 226A and 226B for clarity. More particularly, the adhesive 226A can be disposed about the central portion 206 to couple the flexible member 220 to the central portion 206, and the adhesive 226B can be disposed about the proximal end portion 204 to provide a strain relief for the flexible member 220. Although shown and described as being placed about the surface of the electronic stimulator 200 in multiple discrete locations, in other embodiments, the adhesive 226 can be disposed on the electronic stimulator 200 as a continuous bead of material.

The flexible member 220 can be constructed from any suitable biocompatible material. In some embodiments, the flexible member 220 can be constructed from a material formulated to be soluble when exposed to a bodily tissue, such as the materials described above with reference to the flexible member 120. In other embodiments, the flexible member 220 can be constructed from a non-soluble material, such as silk. The adhesive 226 can be any suitable biocompatible adhesive of the types shown and described above with reference to the adhesive 126.

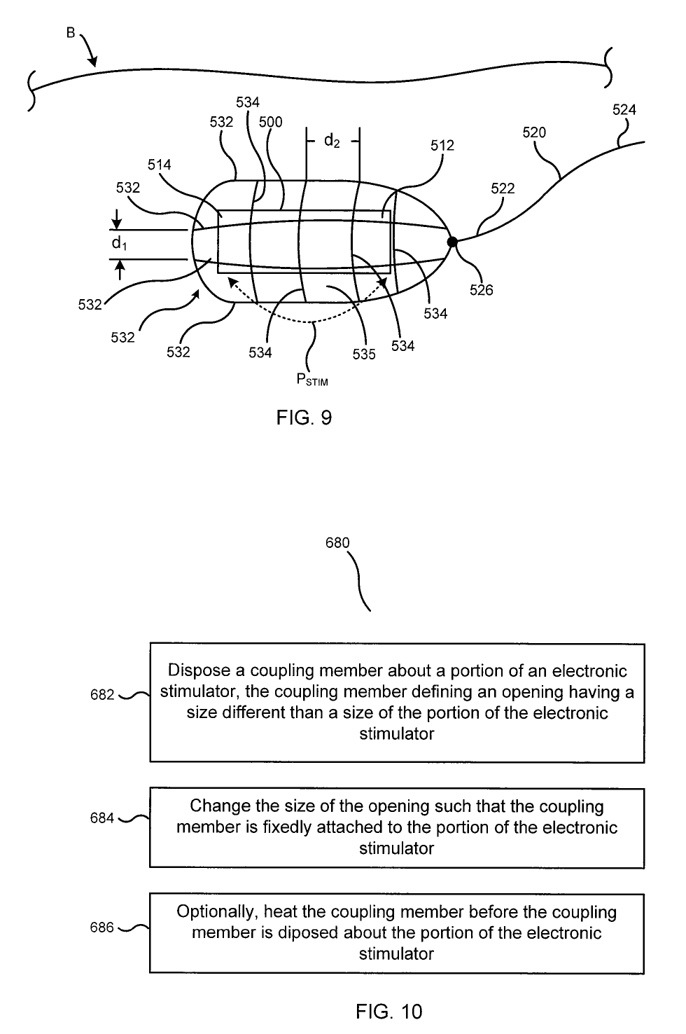

FIG. 9 is a schematic illustration of an enclosure 530 according to an embodiment disposed about an electronic stimulator 500 that is disposed within a body B. The electronic stimulator 500 can be, for example, any implantable stimulator of the types shown and described herein. In some embodiments, for example, the electronic stimulator 500 can be a BION® implantable microstimulator manufactured by Boston Scientific Neuromodulation, a radio frequency-powered implantable microstimulator developed by the Alfred Mann Foundation, a radio frequency-powered implantable microstimulator developed by the Alfred Mann Institute, or the like. The electronic stimulator 500 has a first electrode 512 and a second electrode 514 spaced apart from the first electrode. The first electrode 512 can be either a positively-charged electrode (i.e., an anode) or a negatively-charged electrode (i.e., a cathode). Similarly, the second electrode 514 can be either a positively-charged electrode (i.e., an anode) or a negatively-charged electrode (i.e., a cathode). In this manner, when the electronic stimulator 500 is disposed within the body B, an electronic current can travel between the first electrode 512 and the second electrode 514 along a stimulation path P_{STIM} to stimulate a target location such as a muscle, a nerve or the like.

The enclosure 530 is disposed about the electronic stimulator 500 when the electronic stimulator 500 is disposed within a body B. In this manner, movement of the enclosure 530 results in movement of the electronic stimulator 500 within the body B. Thus, this arrangement allows a user to change the position, orientation and/or location of the electronic stimulator 500 within the body B by moving the enclosure 530. The enclosure 530 is constructed from multiple longitudinal filaments 532 and multiple lateral filaments 534. More particularly, the longitudinal filaments 532 are spaced apart by a first distance d₁, and the lateral filaments 534 are spaced apart by a second distance d₂. Thus, the enclosure 530 defines a plurality of openings 535 defined between the longitudinal filaments 532 and the lateral filaments 534. Note that only one of the openings 535 is labeled in FIG. 9. In this manner, the enclosure 530 is a basket-like or mesh-like structure configured to contain the electronic stimulator 500 while allowing for the flow of bodily tissue, bodily fluids and/or electronic current within the enclosure 530. More particularly, the openings 535 are configured to allow an electronic current to travel along the stimulation path P_{STIM}, which extends from a region within the enclosure 530 to a region outside of the enclosure 530, between the first electrode 512 and the second electrode 514 without the enclosure 530 substantially impeding the flow of the current.

The longitudinal filaments 532 and the lateral filaments 534 can be spaced apart by any suitable distance. In some embodiments, for example, the distance d₁ can be substantially equal to the distance d₂. In other embodiments, the distance d₁ can be different from the distance d₂. In some embodiments, the distance d₁ and/or the distance d₂ can be at least 0.5 millimeters. In other embodiments, the distance d₁ and/or the distance d₂ can be at between approximately 0.5 millimeters and 1.0 millimeters. In yet other embodiments, the longitudinal filaments 532 and the lateral filaments 534 can be spaced apart such that a ratio of the area of the openings 535 to the area of an outer surface (not identified in FIG. 9) of the enclosure 530 is at a desired value. Such a ratio can, for example, be associated with the attenuation of the current flow along the stimulation path P_{STIM}. In some embodiments, for example, the ratio of the area of the openings 535 to the area of an outer surface of the enclosure 530 can be at least 0.5. In other embodiments, for example, the ratio of the area of the openings 535 to the area of an outer surface of the enclosure 530 can be greater than 0.75.

A first end portion 522 of a flexible member 520 is coupled to the electronic stimulator 500 by an adhesive 526. The flexible member 520 includes a second end portion 524 disposed apart from the first end portion 522 (e.g., adjacent a skin incision, outside of the body B, or the like), such that a user can move the electronic stimulator 500 within the body B via the flexible member 520 and the enclosure 530. In some embodiments, for example, a user can remove the electronic stimulator 500 from the body B via the flexible member 520 and the enclosure 530.

At least one filament of the enclosure 530 (i.e., at least one of the longitudinal filaments 532 or at least one of the lateral filaments 534) is constructed from a material formulated to be soluble when exposed to a bodily tissue, as described above. Similarly stated, at least a portion of the enclosure 530 is configured to dissolve after a being disposed within the body B. Said another way, at least a portion of the enclosure 530 is configured to be broken down and/or metabolized by the body B after a being disposed within the body B. The at least one filament can be constructed from any suitable biocompatible material, of the types described herein.

The flexible member 520 can be constructed from any suitable biocompatible material. In some embodiments, the flexible member 520 can be constructed from a material formulated to be soluble when exposed to a bodily tissue, such as the materials described above with reference to the flexible member 120. In other embodiments, the flexible member 520 can be constructed from a non-soluble material, such as silk. The adhesive 526 can be any suitable biocompatible adhesive of the types shown and described above with reference to the adhesive 126.

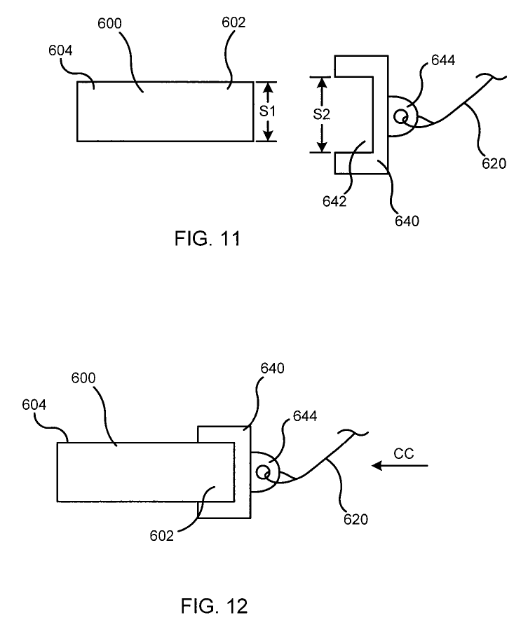

Although the flexible member 120 is shown and described above as being coupled to the electronic stimulator 100 by the adhesive 126, in other embodiments, a flexible member can be coupled to an electronic stimulator by a coupling member that is coupled to the electronic stimulator. For example, FIG. 10 is a flow chart of a method 680 of attaching a coupling member to an electronic stimulator according to an embodiment. The method illustrated in FIG. 10 is discussed with reference to FIGS. 11 and 12, which are schematic illustrations of an electronic stimulator 600 and a coupling member 640 in a first configuration and a second configuration, respectively. The method includes disposing a coupling member about a portion of an electronic stimulator, 682. Referring to FIG. 11, the electronic stimulator 600, which can be any suitable implantable electronic stimulator described herein, includes a first end portion 602, a second end portion 604 and a central portion 606 therebetween. Each of the first end portion 602 and the second end portion 604 can include an electrode, as described above. The first end portion 602 has a size S1, which can be, for example a diameter of the first end portion 602.

The coupling member 640 defines an opening 642 and includes an eyelet 644. The eyelet 644 is configured to be coupled to a flexible member 620. The flexible member 620 can be any suitable flexible member of the types shown and described herein. The flexible member 620 can be coupled to the eyelet 644 in any suitable manner, such as for example, by tying the flexible member 620 to the eyelet 644, by adhesively coupling the flexible member 620 within the opening defined by the eyelet 644, and/or the like.

The opening 642 has a size S2, which can be, for example a diameter of the opening 642. In other embodiments, however, the opening 642 can have a non-circular cross-section. The size S2 of the opening 642 is different than the size SI of the first end portion 602 of the electronic stimulator. As shown by the arrow CC in FIG. 12, the coupling member 640 can be disposed about the first end portion 602 of the electronic stimulator 600. Similarly stated, the first end portion 602 of the electronic stimulator 600 is disposed within the opening 642 of the coupling member 640.

Returning to the flow chart shown in FIG. 10, the size of the opening is changed such that the coupling member is fixedly attached to the portion of the electronic stimulator, 684. Referring to FIGS. 11 and 12, in some embodiments, the size S2 of the opening 642 can be greater than the size S1 of the first end portion 602 before the coupling member 640 is disposed about the electronic stimulator 600 and can be changed such that the size S2 of the opening 642 is less than the size SI of the first end portion 602 after the coupling member 640 is disposed about the electronic stimulator 600. In this manner, the interference fit (i.e., the frictional fit) between the opening 642 of the coupling member 640 and the first end portion 602 fixedly couples the coupling member 640 to the electronic stimulator 600. In some embodiments, for example, the coupling member 640 can be constructed from a material formulated to decrease in size when exposed to heat (e.g., a “heat-shrink” material). Such materials can include polymeric materials having at least a 2:1 shrink ratio when exposed to a temperature of at least 121 degrees Celsius (250 degrees Fahrenheit). Such materials can include, for example, HS-714 available from Insultab, Inc. In such embodiments, the coupling member 640 can be disposed about the first end portion 602 of the electronic stimulator 600 and then exposed to a heat source to reduce the size S2 of the opening 642.

Although such “heat-shrink” materials are often formulated to irreversibly change size when exposed to heat, in some embodiments, the size of the coupling member can be reversibly changed. Referring to the flow chart shown in FIG. 10, in some embodiments, the method can optionally include heating the coupling member before the coupling member is disposed about the portion of the electronic stimulator, 686. Referring to FIGS. 11 and 12, in such embodiments, the nominal size S2 of the opening 642 (i.e., the size of the opening 642 when the coupling member 640 is at room temperature or body temperature) is less than the size S1 of the first end portion 602. Heating the coupling member 640 causes the coupling member 640 to thermally expand, thereby reversibly increasing the size S2 of the opening 642. In this manner, the size S2 of the opening 642 can be changed before the coupling member 640 is disposed about the electronic stimulator 600 such that the size S2 of the opening 642 is greater than the size S1 of the first end portion 602. After the coupling member 640 is disposed about the electronic stimulator 600, the coupling member 640 can be cooled (either by removing the source of heat and/or by actively cooling the coupling member 640) such that the size S2 of the opening 642 returns to its nominal value. The contraction of the opening 642 about the first portion 602 results in an interference fit between the opening 642 of the coupling member 640 and the first end portion 602, thereby fixedly coupling the coupling member 640 to the electronic stimulator 600.

The coupling member 640 can be constructed of any suitable material, and the opening 642 can have any suitable size S2 that results in a desired interference fit when the coupling member 640 is heated to a predetermined temperature, disposed about the first end portion 602, and subsequently cooled. For example, in some embodiments, the nominal size S1 of the first end portion 602 of the electronic stimulator 600 can be a diameter of approximately 3.1 millimeters. The nominal size S2 of the opening can be a diameter of approximately 3.21 millimeters, thereby resulting in an interference fit of approximately 0.11 millimeters. As described above, the coupling member 640 can be heated such that the size S2 of the opening increases to approximately 3.26 millimeters (i.e., an increase of approximately 0.16 millimeters), thereby resulting in a diametral clearance of approximately 0.05 millimeters. The coupling member 640 can then be disposed about the first end portion 602 of the electronic stimulator 600 and subsequently cooled such that the coupling member 640 is fixedly coupled to the electronic stimulator 600. In some embodiments, for example, the coupling member can be constructed of a polymer having a thermal expansion coefficient of approximately 9.5×10−5 mm/mm/° C. Such materials can include, for example, ABS (acrylonitrile, butadiene styrene). In such embodiments, the coupling member 640 can be heated such that the temperature of the coupling member 640 increases approximately 173 degrees Celsius to achieve the change in the size S2 as described above.

In some embodiments, the size S2 of the opening 642 can be less than the size S1 of the first end portion 602 before the coupling member 640 is disposed about the electronic stimulator 600 and can be changed during and/or after the coupling member 640 is disposed about the electronic stimulator 600. For example, in some embodiments, at least a portion of the coupling member 640 can be constructed from an elastic material. When the coupling member 640 is disposed about the first end portion 602, the portion of the coupling member 640 can be elastically deformed (i.e., stretched) such that the first end portion 602 can be received within the opening 642. In this manner, a coupling force can be produced by the elastic properties of the coupling member 650 to fixedly couple the coupling member 640 to the first end portion 602 of the electronic stimulator 600. Such elastic materials can include any suitable silicone elastomer, such as, for example, Silastic® produced by Dow Corning Corp.

Although the coupling member 640 is shown and described above as being fixedly coupled to the first end portion 602 of the electronic stimulator 600, in other embodiments, a coupling member can be coupled to any suitable portion of an electronic stimulator. For example, in some embodiments, a coupling member can be coupled to a central portion of an electronic stimulator.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above. Thus, the breadth and scope of the invention should not be limited by any of the above-described embodiments. While the invention has been particularly shown and described with reference to specific embodiments thereof, it will be understood that various changes in form and details may be made.

For example, although the proximal end portion 124 of the flexible member 120 is shown as being disposed beneath the skin S, in other embodiments, the flexible member 120 can have length such that the proximal end portion 124 is disposed outside of the body B when the electronic stimulator 100 is disposed within the body B.

Although the flexible members are shown and described above as being constructed from a material that is soluble when exposed to a bodily tissue, in other embodiments, a flexible member can be constructed from more than multiple different materials. In some embodiments, for example, a flexible member can include an electrically conductive material. In other embodiments, a flexible member can include a radio-opaque material. In yet other embodiments, a flexible member can include a non-soluble material.

Although the flexible member 120 is shown and described above as being coupled to an outer surface of the electronic stimulator 100, in some embodiments, a flexible member can be coupled to any suitable portion of an electronic stimulator. For example, in some embodiments, a flexible member can be coupled within an interior portion of an electronic stimulator. Such an interior portion can include, for example, a lumen and/or a drilling within a casing of the electronic stimulator. In such embodiments, an adhesive can be disposed within the interior portion of the electronic stimulator to couple the flexible member to the electronic stimulator.

Although the enclosure 530 is shown and described above as being constructed from multiple filaments (i.e., longitudinal filaments 532 and lateral filaments 534), in some embodiments, an enclosure can be constructed from a single filament. For example, in some embodiments, a single filament can be wound and/or weaved to produce an enclosure.

Although flexible member 520 is shown and described above as being coupled to the enclosure 530 by the adhesive 526, in some embodiments, the flexible member 520 can be coupled to the enclosure 530 by any suitable method. Such methods can include, for example, tying, melt bonding, or the like. In some embodiments, the flexible member 520 and the enclosure 530 can be monolithically constructed. Similarly stated, in some embodiments, the flexible member 520 and at least a portion of the enclosure 530 can be constructed a single filament.

Although the enclosure 530 is shown and described above as being constructed from flexible filaments, in some embodiments, an enclosure can be constructed from elastic filaments. In this manner, the enclosure can be stretched to fit about the electronic stimulator.

Although various embodiments have been described as having particular features and/or combinations of components, other embodiments are possible having a combination of any features and/or components from any of embodiments where appropriate. For example, in some embodiments, the enclosure 530 and the flexible member 520 can be configured to withstand the tensile forces as described above with reference to the flexible member 120.

What is claimed is:

1. An apparatus, comprising:
an electronic stimulator configured to be implanted within a body; and
a flexible member coupled to the electronic stimulator by an adhesive.

2. The apparatus of claim 1, wherein a distal end portion of the flexible member is coupled to an outer surface of the electronic stimulator.

3. The apparatus of claim 1, wherein a distal end portion of the flexible member is wrapped about an outer surface of the electronic stimulator.

4. The apparatus of claim 1, wherein a distal end portion of the flexible member is wrapped about an outer surface of the electronic stimulator by at least two full revolutions.

5. The apparatus of claim 1, wherein a distal end portion of the flexible member is coupled to an outer surface of the electronic stimulator, the flexible member having a length such that a proximal end portion of the flexible member is disposed outside of the body when the electronic stimulator is disposed within the body.

6. The apparatus of claim 1, wherein the flexible member includes an electrically conductive material.

7. The apparatus of claim 1, wherein:
the electronic stimulator includes a cathode electrode and an anode electrode; and
a distal end portion of the flexible member is coupled to the anode electrode.

8. The apparatus of claim 1, wherein the flexible member is formulated to be soluble when exposed to a bodily tissue

9. The apparatus of claim 1, wherein the adhesive is formulated to be soluble when exposed to a bodily tissue.

10. The apparatus of claim 1, wherein:
the flexible member is formulated to substantially dissolve within fourteen days of contact with the bodily tissue; and
the adhesive is formulated to substantially dissolve within fourteen days of contact with the bodily tissue.

11. The apparatus of claim 1, wherein:
the flexible member and the adhesive are collectively configured to withstand tensile force of at least 1.8 N applied to a proximal end portion of the flexible member.

12. The apparatus of claim 1, wherein the electronic stimulator is devoid of an eyelet.

13. A method, comprising:
inserting at least a distal end portion of an insertion tool within a body such that a stimulation portion of an electronic implant is disposed within the body and at least a proximal portion of the electronic implant is disposed outside the body, the proximal portion being soluble in a bodily fluid, the proximal portion being coupled to the stimulation portion by an adhesive;
removing the insertion tool from the body after the inserting such that the stimulation portion is at a first location within the body; and
moving the proximal portion of the electronic implant via the proximal portion of the electronic implant from a region outside the body such that the stimulation portion is moved to a second location within the body, the second location being different from the first location.

14. The method of claim 13, wherein the proximal portion of the electronic implant is configured to substantially dissolve within a predetermined time of being in contact with the bodily fluid.

15. The method of claim 13, wherein the inserting includes inserting the distal end portion of the insertion tool such that the stimulation portion of the electronic implant is disposed apart from a nerve by a predetermined distance.

16. The method of claim 13, wherein the inserting includes inserting the distal end portion of the insertion tool such that the stimulation portion of the electronic implant is adjacent a nerve within the body and a terminal portion of the electronic implant is beneath the skin, the terminal portion of the electronic implant coupled to the stimulation portion of the electronic implant by a substantially flexible conductor.

17. An apparatus, comprising:
an enclosure configured to be disposed about an electronic stimulator when the electronic stimulator is disposed within a body, the enclosure constructed from at least one filament formulated to be soluble when exposed to a bodily tissue; and
a flexible member coupled to the enclosure, the flexible member having a length such that a proximal end portion of the flexible member is disposed outside of the body when the electronic stimulator is disposed within the body.

18. The apparatus of claim 17, wherein
the enclosure is configured to be disposed about the electronic stimulator such that movement of the proximal end portion of the flexible member results in movement of the enclosure and the electronic stimulator within the body.

19. The apparatus of claim 17, wherein the enclosure is constructed from a plurality of filaments coupled together such that a first filament from the plurality of filaments is spaced apart from a second filament from the plurality of filaments by at least 0.5 millimeters.

20. The apparatus of claim 17, wherein the enclosure includes an outer surface having a plurality of openings, a ratio of an area of the openings to an area of the outer surface being at least 0.5.

21. The apparatus of claim 17, wherein the flexible member is coupled to the enclosure by an adhesive.

22. The apparatus of claim 17, wherein the flexible member is a suture formulated to be soluble when exposed to the bodily tissue.

23. A method, comprising:
disposing a coupling member about a portion of an electronic stimulator, the coupling member defining an opening having a size different than a size of the portion of the electronic stimulator before the disposing; and
changing the size of the opening of the coupling member such that the coupling member is fixedly attached to the portion of the electronic stimulator.

24. The method of claim 23, wherein:
the size of the opening is greater than the size of the portion of the electronic stimulator; and
the changing includes reducing the size of the of the opening of the coupling member such that the size of the opening of the coupling member is less than the size of the portion of the electronic stimulator.

25. The method of claim 23, wherein the changing includes heating the coupling member after the disposing.

26. The method of claim 23, further comprising:
heating the coupling member before the disposing,
the changing including cooling the coupling member after the disposing.

27. The method of claim 23, further comprising:
applying a source of heat to the coupling member before the disposing such that the temperature of at least a portion of the coupling member increases by at least 173 degrees Celsius,
the changing including removing the source of heat from the coupling member after the disposing.

28. The method of claim 23, wherein:
the size of the opening of the coupling member is a diameter; and
the changing includes reducing the diameter of the opening of the coupling member by approximately 0.16 millimeters.
